(19) United States
(12) Patent Application Publication
(10) Pub. No.: US 2020/0048641 A1
(43) Pub. Date: Feb. 13, 2020

(54) METHODS FOR ENHANCING YIELD OF RECOMBINANT ADENO-ASSOCIATED VIRUS

(71) Applicant: Ultragenyx Pharmaceutical Inc., Novato, CA (US)

(72) Inventors: Ying Jing, Reinach (CH); Jan Panteli, Cambridge, MA (US); Kelly Reed Clark, Westerville, OH (US)

(21) Appl. No.: 16/495,974

(22) PCT Filed: Mar. 22, 2018
(86) PCT No.: PCT/US2018/023839

§ 371 Date: Sep. 20, 2019

Related U.S. Application Data
(60) Provisional application No. 62/474,932, filed on Mar. 22, 2017.

(51) Int. Cl. C12N 15/64; C12N 15/86; C12N 7/00

(52) U.S. Cl. CPC C12N 15/64; C12N 15/86; C12N 2750/14151; C12N 2750/14141; C12N 7/00

(57) ABSTRACT

The invention provides methods for the production of recombinant adeno-associated virus vectors (rAAV), e.g., comprising the addition of a surfactant, e.g., Triton X-100, to a producer cell culture medium.

### CROSS-REFERENCE TO RELATED APPLICATIONS

This application claims the benefit of and priority to U.S. application No. 62/474,932 filed Mar. 22, 2017, the entire content of which is hereby incorporated by reference in their entirety for all purposes.

### FIELD OF THE INVENTION

The invention relates generally to methods for enhancing yield of recombinant adeno-associated virus (rAAV), and, more particularly, the invention relates to the use of surfactants in cell culture media to enhance rAAV yield.

### BACKGROUND

Adeno-associated virus (AAV) is a non-pathogenic, replication-defective parvovirus. Recombinant AAV vectors (rAAV) have many unique features that make them attractive as vectors for gene therapy. In particular, rAAV vectors can deliver therapeutic genes to dividing and nondividing cells, and these genes can persist for extended periods without integrating into the genome of the targeted cell. Given the widespread therapeutic applications of rAAV, there exists an ongoing need for improved methods of rAAV vector production including methods to achieve larger vector batches, higher vector titer, and improved vector quality and safety.

Current manufacturing methods for rAAV typically employ clarification of rAAV products by filtration for the removal of cells, cell debris, insoluble precipitants, aggregates, and other materials found in producer cell cultures. However, such clarification has been observed to result in significant loss of rAAV yield.

### SUMMARY OF THE INVENTION

Provided herein are methods for enhancing yield, e.g., clarification yield, of recombinant adeno-associated virus (rAAV). The methods comprise, e.g., the addition of surfactants, e.g., Triton X-100, to a cell culture medium. In one aspect, the invention provides a method of producing a clarified recombinant adeno-associated virus vector (rAAV) product comprising the steps of: incubating a plurality of producer cells in a cell culture medium under conditions that promote release of rAAV particles, whereby rAAV particles are released from the producer cells into the culture medium, and the producer cells are not substantially lysed; recovering the cell culture medium from the producer cells, so that the recovered cell culture medium is substantially free of intact producer cells and contains rAAV particles; adding a surfactant to the recovered cell culture medium, wherein the surfactant is a non-ionic surfactant with a hydrophilic-lipophilic balance (HLB) between 12 and 15; and/or passing the recovered cell culture medium containing surfactant through a first filter. In certain embodiments, the method does not comprise a step of lysing the producer cells.

In certain embodiments, the step of recovering the cell culture medium from the producer cells, so that the recovered cell culture medium is substantially free of intact producer cells and contains rAAV particles, comprises the steps of centrifuging the producer cells in the cell culture medium to create a cell pellet, separating the cell culture medium from the cell pellet, and retaining the cell culture medium. The producer cells may, e.g., be centrifuged at a speed of greater than 150×g or 300×g, or at a speed of less than 500×g or 13,000×g.

In certain embodiments, the step of recovering the cell culture medium from the producer cells, so that the recovered cell culture medium is substantially free of intact producer cells and contains rAAV particles, further comprises aspirating the cell culture medium into a vessel or of changing the pH of the cell culture medium.

In another aspect, the invention provides a method of producing a clarified recombinant adeno-associated virus vector (rAAV) product comprising the steps of: incubating a plurality of producer cells in a cell culture medium under conditions that promote release of rAAV particles, whereby rAAV particles are released from the producer cells into the culture medium, and the producer cells are not substantially lysed; adding a surfactant to the cell culture medium, wherein the surfactant is a non-ionic surfactant with an HLB between 12 and 15; incubating the producer cells in the surfactant-containing cell culture medium for a time, the time being insufficient to cause the lysis of 50% of the producer cells; and/or passing the cell culture medium containing the surfactant through a first filter.

In certain embodiments, incubation of the producer cell in the surfactant-containing cell culture medium results in lysis of less than 40% of the producer cells, less than 30% of the producer cells, less than 20% of the producer cells, less than the 10% of producer cells, or less than 1% of the producer cells.

In any of the foregoing methods, release of the rAAV particles from the producer cells into the culture medium may, e.g., result in lysis of less than 50% of the producer cells, less than 30% of the producer cells, less than 20% of the producer cells, less than 10% of the producer cells, or less than 1% of the producer cells.

In any of the foregoing methods, the filter may, e.g., be a PES membrane filter, a PVDF membrane filter, a nylon membrane filter, or a MCE membrane filter. In certain embodiments, the filter is a PES membrane filter. The filter may, e.g., have a pore size of less than 1 μm, a pore size of less than 0.5 μm, or a pore size of 0.22 μm. In certain embodiments, the recovered cell culture medium is passed through a first filter. In certain embodiments, the recovered cell culture medium is passed through multiple filters, e.g., a first filter and a second filter.

In any of the foregoing methods, the surfactant may, e.g., comprise a non-ionic surfactant with an HLB between 12 and 15. The surfactant may be, e.g., an octylphenol ethoxylate. In certain embodiments, the surfactant is an octylphenol ethoxylate with an HLB between 13 and 14, an octylphenol ethoxylate with an average polyethylene oxide chain length between 8 and 12, or an octylphenol ethoxylate with an average polyethylene oxide chain length between 9 and 10.

In any of the foregoing methods, the surfactant may, e.g., be added to the cell culture medium at a final concentration of about 0.1% to about 0.5%, about 0.1% to about 0.3%, about 0.5% to about 1%, or about 0.7% to about 1%. In certain embodiments, the surfactant is added to the cell culture medium at a final concentration of about 1%. In certain embodiments, the surfactant is added to the cell culture medium at a final concentration of about 0.1%.

In any of the foregoing methods, the concentration of rAAV particles in the rAAV-containing cell culture medium after the first filtration step may, e.g., be greater than 80%, 90%, 95%, or 99% of the concentration of rAAV particles in the rAAV-containing cell culture medium immediately prior to the first filtration step.

It is contemplated that the producer cell may be a mammalian cell, for example, a HeLa, HEK293, COS, A549, or Vero cell. It is also contemplated that the producer cell may be an insect cell, for example, a Sf9, Sf-21, Tn-368, or BTI-Tn-5B1-4 cell. In certain embodiments, the producer cell is a HeLa cell. In certain embodiments, the producer cell is a HEK293 cell. The producer cells may include a heterologous nucleotide sequence flanked by AAV inverted terminal repeats; rep and cap gene functions; and/or helper viral functions. In certain embodiments, the producer cell comprises a heterologous nucleotide sequence flanked by AAV inverted terminal repeats; rep and cap genes; and helper virus genes.

In other aspects, the invention provides a rAAV produced by any of the contemplated methods, or a composition comprising a rAAV produced by any of the contemplated methods.

These and other aspects and features of the invention are described in the following detailed description and claims.

### BRIEF DESCRIPTION OF THE DRAWINGS

The invention can be more completely understood with reference to the following drawings.

FIG. 1 is a bar graph showing rAAV titer (GC/ml as measured by qPCR) in cell culture supernatants before and after filtration in the presence or absence of 1% Triton X-100. n=3 for all experiments, and error bars represent standard error.

FIG. 2 is a bar graph showing rAAV titer (GC/ml measured by qPCR) in cell culture bulk harvest material before and after filtration in the presence or absence of Triton X-100. rAAV titers (GC/ml measured by qPCR) in cell culture pellets were also measured to determine intracellular rAAV. n=3 for all experiments, and error bars represent standard error.

FIG. 3 is a bar graph showing rAAV titer (GC/ml measured by qPCR) in cell culture bulk harvest material before and after filtration in the presence or absence of 0%, 0.01%, 0.1% or 1% Triton X-100. Error bars represent standard deviation of triplicate measurements of a single sample (n=1).

FIG. 4 is a bar graph showing rAAV titer (GC/ml measured by qPCR) in cell culture supernatants before and after multiple filtration steps in the presence or absence of Triton X-100. n=3 for all experiments, and error bars represent standard error.

### DETAILED DESCRIPTION

The invention is based, in part, upon the discovery that addition of a surfactant, e.g., Triton X-100, to a recombinant adeno-associated virus (rAAV) producer cell culture medium can increase the recovery of rAAV from the producer cell culture medium. In particular, addition of a surfactant, e.g., Triton X-100, can, e.g., reduce the loss of rAAV particles during subsequent filtration steps.

The surfactant may, for example, be added to the cell culture medium following separation of the cell culture medium from the producer cells, i.e., recovery of the cell culture medium from the producer cells, e.g., by centrifugation. Accordingly, in one aspect, the invention provides a method of producing a clarified recombinant adeno-associated virus vector (rAAV) product. The method includes the steps of: incubating a plurality of producer cells in a cell culture medium under conditions that promote release of rAAV particles, whereby rAAV particles are released from the producer cells into the culture medium, and the producer cells are not substantially lysed; recovering the cell culture medium from the producer cells, so that the recovered cell culture medium is substantially free of intact producer cells and contains rAAV particles; adding a surfactant to the recovered cell culture medium, wherein the surfactant is a non-ionic surfactant with a hydrophilic-lipophilic balance (HLB) between 12 and 15; and/or passing the recovered cell culture medium containing surfactant through a first filter. In certain embodiments, the method does not comprise a step of lysing the producer cells.

In certain embodiments, the step of recovering the cell culture medium from the producer cells, so that the recovered cell culture medium is substantially free of intact producer cells and contains rAAV particles, comprises centrifuging the producer cells in the cell culture medium to create a cell pellet, separating the cell culture medium from the cell pellet, and retaining the cell culture medium. The producer cells may, e.g., be centrifuged at a speed of greater than 150×g or 300×g. The producer cells may, e.g., be centrifuged at a speed of less than 500×g or 13,000×g. The producer cells may, e.g., be centrifuged at a speed of less than 14,000×g or 15,000×g. The producer cells may, e.g., be centrifuged at a speed of about 13,000×g. In certain embodiments, the step of recovering the cell culture medium from the producer cells, so that the recovered cell culture medium is substantially free of intact producer cells and contains rAAV particles, comprises aspirating the cell culture medium into a vessel or changing the pH of the cell culture medium.

The surfactant may also, for example, be added prior to recovery of the cell culture medium from the producer cell, but in amount insufficient to cause substantial lysis of the producer cell. Thus, in another aspect, the invention provides a method of producing a clarified recombinant adeno-associated virus vector (rAAV) product comprising the steps of: incubating a plurality of producer cells in a cell culture medium under conditions that promote release of rAAV particles, whereby rAAV particles are released from the producer cells into the culture medium, and the producer cells are not substantially lysed; adding a surfactant to the cell culture medium, wherein the surfactant is a non-ionic surfactant with an HLB between 12 and 15; incubating the producer cells in the surfactant-containing cell culture medium for a time, the time being insufficient to cause the lysis of 50% of the producer cells; and/or passing the cell culture medium containing surfactant through a first filter.

In certain embodiments, release of the rAAV particles from the producer cells into the culture medium results in lysis of less than the 50% of the producer cells, less than the 40% of the producer cells, less than 30% of the producer cells, less than the 20% of the producer cells, less than 10% of the producer cells, less than 5% of the producer cells, or less than 1% of the producer cells.

In certain embodiments, incubation of the producer cells in the surfactant-containing cell culture medium results in lysis of less than 40% of the producer cells, less than 30% of the producer cells, less than 20% of the producer cells, less than 10% of the producer cells, less than 5% of the producer cells, or less than 1% of the producer cells.

The cell culture medium, e.g., the recovered cell culture medium, may be incubated with the surfactant for any appropriate time. The incubation time may vary, for example, depending upon the surfactant, the concentration of the surfactant, the temperature, and the producer cell type. In certain embodiments, the producer cells are incubated with the surfactant for around 1 minute, around 5 minutes, around 10 minutes, around 15 minutes, around 30 minutes, around 45 minutes, around 60 minutes, around 2 hours, around 3 hours, around 4 hours, around 5 hours, around 6 hours, around 7 hours, or around 8 hours, or for more than 8 hours.

In certain embodiments, the producer cells, e.g., Hela cells, are incubated with surfactant, e.g., Triton X-100, for around 1 hour.

Lysis of cells, e.g., producer cells, may be assayed by any method known in the art, for example, membrane integrity assays, trypan blue exclusion assays, examination under optical microscopy, or, staining with a stain that can distinguish live and dead cells and subsequent analysis by flow cytometry.

Exemplary filters for use in methods of the invention include PES membrane, PVDF membrane, nylon membrane, or MCE membrane filters. In certain embodiments, the filter is a PES membrane filter. The filter may have a pore size of less than 1 μm, less than 0.5 μm, e.g., 0.22 μm. In certain embodiments, the recovered cell culture medium is passed through a single filter, e.g., a first filter. In certain embodiments, the recovered cell culture medium is passed through multiple filters, e.g., a first filter and a second filter.

Various features and aspects of the invention are discussed in more detail below.

### I. Adeno-Associated Virus

Adeno-associated virus (AAV) is a small, nonenveloped icosahedral virus of the genus Dependoparvovirus and family Parvovirus. AAV has a single-stranded linear DNA genome of approximately 4.7 kb. AAV includes numerous serologically distinguishable types including serotypes AAV-1 to AAV-12, as well as more than 100 serotypes from nonhuman primates (See, e.g., Srivastava (2008) J. Cell Biochem., 105(1): 17-24, and Gao et al. (2004) J. Virol., 78(12), 6381-6388). Any AAV type may be used in the methods of the present invention. AAV is capable of infecting both dividing and quiescent cells of several tissue types, with different AAV serotypes exhibiting different tissue tropism. AAV is non-autonomously replicating, and has a life cycle with a latent phase and an infectious phase. In the latent phase, after a cell is infected with an AAV, the AAV site-specifically integrates into the host's genome as a provirus. The infectious phase does not occur unless the cell is also infected with a helper virus (for example, adenovirus (AV) or herpes simplex virus), which allows the AAV to replicate.

The wild-type AAV genome contains two 145 nucleotide inverted terminal repeats (ITRs), which contain signal sequences directing AAV replication, genome encapsidation and integration. In addition to the ITRs, three AAV promoters, p5, p19, and p40, drive expression of two open reading frames encoding rep and cap genes. Two rep promoters, coupled with differential splicing of the single AAV intron, result in the production of four rep proteins (Rep 78, Rep 68, Rep 52, and Rep 40) from the rep gene. Rep proteins are responsible for genomic replication. The Cap gene is expressed from the p40 promoter, and encodes three capsid proteins (VP1, VP2, and VP3) which are splice variants of the cap gene. These proteins form the capsid of the AAV particle.

Because the cis-acting signals for replication, encapsidation, and integration are contained within the ITRs, some or all of the 4.3 kb internal genome may be replaced with foreign DNA, for example, an expression cassette for an exogenous protein of interest. In this case the rep and cap proteins are provided in trans on, for example, a plasmid. In order to produce an AAV vector, a producer cell line permissive of AAV replication must express the rep and cap genes, the ITR-flanked expression cassette, and helper functions provided by a helper virus, for example AV genes E1a, E1b55K, E2a, E4orf6, and VA (Weitzman et al. (2011) Adeno-associated virus biology. Adeno-Associated Virus: Methods and Protocols, pp. 1-23). Production of AAV vector can also result in the production of helper virus particles, which must be removed or inactivated prior to use of the AAV vector. Numerous cell types are suitable for producing AAV vectors, including HEK293 cells, COS cells, HeLa cells, BHK cells, Vero cells, as well as insect cells (See, e.g., U.S. Pat. Nos. 6,156,303, 5,387,484, 5,741,683, 5,691,176, 5,688,676, and 8,163,543, U.S. Publication No. 20020081721, and PCT Publication Nos. WO00/47757, WO00/24916, and WO96/17947). AAV vectors are typically produced in these cell types by one plasmid containing the ITR-flanked expression cassette, and one or more additional plasmids providing the additional AAV and helper virus genes.

AAV of any serotype may be used in the present invention. Similarly, it is contemplated that any AV type may be used, and a person of skill in the art will be able to identify AAV and AV types suitable for the production of their desired recombinant AAV vector (rAAV). AAV and AV particles may be purified, for example by affinity chromatography, iodixonal gradient, or CsCl gradient.

The genome of wild-type AAV is single-stranded DNA and is 4.7 kb. AAV vectors may have single-stranded genomes that are 4.7 kb in size, or are larger or smaller than 4.7 kb, including oversized genomes that are as large as 5.2 kb, or as small as 3.0 kb. Further, vector genomes may be substantially self-complementary, so that within the virus the genome is substantially double stranded. AAV vectors containing genomes of all types are suitable for use in the method of the instant invention.

As discussed above, AAV requires co-infection with a helper virus in order to enter the infectious phase of its life cycle. Helper viruses include Adenovirus (AV), and herpes simplex virus (HSV), and systems exist for producing AAV in insect cells using baculovirus. It has also been proposed that papilloma viruses may also provide a helper function for AAV (See, e.g., Hermonat et al., Molecular Therapy 9, S289-S290 (2004)). Helper viruses include any virus capable of creating an allowing AAV replication. AV is a nonenveloped nuclear DNA virus with a double-stranded DNA genome of approximately 36 kb. AV is capable of rescuing latent AAV provirus in a cell, by providing E1a, E1b55K, E2a, E4orf6, and VA genes, allowing AAV replication and encapsidation. HSV is a family of viruses that have a relatively large double-stranded linear DNA genome encapsidated in an icosahedral capsid, which is wrapped in a lipid bilayer envelope. HSV are infectious and highly transmissible. The following HSV-1 replication proteins were identified as necessary for AAV replication: the helicase/primase complex (UL5, ULB, and UL52) and the DNA binding protein ICP8 encoded by the UL29 gene, with other proteins enhancing the helper function.

2. Production of rAAV

As used herein, the term “producer cell” refers to any cell or cells capable of producing a recombinant adeno-associated virus vector (rAAV). In certain embodiments, the producer cell is a mammalian cell, for example, a HeLa cell, COS cell, HEK293 cell, A549 cell, BHK cell, or Vero cell. In certain embodiments, the producer cell is an insect cell, for example, a Sf9 cell, Sf-21 cell, Tn-368 cell, or BTI-Tn-5B1-4 (High-Five) cell. Unless otherwise indicated, the terms “cell” or “cell line” are understood to include modified or engineered variants of the indicated cell or cell line. A rAAV may be produced from a producer cell using any suitable method known in the art.

As discussed above, to allow for production of rAAV, the producer cell must be provided with AAV inverted terminal repeats (ITRs) which may, for example, flank a heterologous nucleotide sequence of interest, AAV rep and cap gene functions, and additional helper functions. These may be provided to the producer cell using any number of appropriate plasmids or vectors. Additional helper functions can be provided by, for example, an adenovirus (AV) infection, by a plasmid that carries all of the required AV helper function genes, or by other viruses such as HSV or baculovirus. Any genes, gene functions, or other genetic material necessary for rAAV production by the producer cell may transiently exist within the producer cell, or be stably inserted into the producer cell genome. In certain embodiments, the producer cell comprises AAV rep and cap gene functions and a rAAV vector genome. In certain embodiments, the producer cell comprises AAV rep and cap gene functions and at the time of production is provided a rAAV vector genome by a separate recombinant virus. rAAV production methods suitable for use with the methods of the current invention include those disclosed in Clark et al. (1995) Human Gene Therapy 6:1329-1341, Martin et al. (2013) Human Gene Therapy Methods 24:253-269, Thorne et al. (2009) Human Gene Therapy 20:707-714, Fraser Wright (2009) Human Gene Therapy 20:698-706, and Virag et al. (2009) Human Gene Therapy 20:807-817.

### 3. Surfactants

Surfactants are typically amphiphilic compounds that lower the surface tension between two liquids or between a liquid and a solid. In certain embodiments, the surfactant is a non-ionic surfactant with a hydrophilic-lipophilic balance (HLB) between 12 and 15, e.g., between 12 and 15, between 12 and 14, between 12 and 13, between 13 and 15, between 13 and 14, or between 14 and 15. Exemplary non-ionic surfactants with a HLB between 12 and 15 include TWEEN® 60 nonionic detergent, PPG-PEG-PPG Pluronic® 10R5, Polyoxyethylene (18) tridecyl ether, Polyoxyethylene (12) tridecyl ether, MERPOL® SH surfactant, MERPOL® OJ surfactant, MERPOL® HCS surfactant, IGEPAL® CO-720, IGEPAL® CO-630, IGEPAL® CA-720, Brij® S20, Brij® S10, Brij® 010, Brij® C10, BRIJ® 020, ECOSURF EH-9®, ECOSURF EH-14®, TERGITOL 15-S-7®, ECOSURF SA-15® TERGITOL 15-S-9®, TERGITOL 15-S-12®, TERGITOL L-64®, TERGITOL NP-7®, TERGITOL NP-8®, TERGITOL NP-9®, TERGITOL NP-9.5®, TERGITOL NP-10®, TERGITOL NP-11®, TERGITOL NP-12®, TERGITOL NP-13®, TRITON CA®, TRITON RW-50®, TRITON X-114®, and TRITON X-102®. Exemplary surfactants are described, e.g., in Van Os (1998) Nonionic Surfactants: Organic Chemistry, Marcel Dekker, Inc. In certain embodiments, the surfactant is an octylphenol ethoxylate, e.g. an octylphenol ethoxylate with an HLB between 13 and 14, an octylphenol ethoxylate with an average polyethylene oxide chain length between 8 and 12, or an octylphenol ethoxylate with an average polyethylene oxide chain length between 9 and 10. In certain embodiments, the surfactant is Triton X-100.

In certain embodiments, the final concentration of the surfactant in the culture medium is about 0.1% to about 1.0%, about 0.1% to about 0.7%, about 0.1% to about 0.5%, about 0.1% to about 0.3%, about 0.3% to about 1.0%, about 0.3% to about 0.7%, about 0.3% to about 0.5%, about 0.5% to about 1.0%, about 0.5% to about 0.7%, or about 0.7% to about 1.0%. In certain embodiments the final concentration of the surfactant in the culture medium is less than 0.1%, 0.3%, 0.5%, 0.7%, or 1.0%. In certain embodiments the final concentration of the surfactant in the culture medium is about 0.1% to about 0.5%, about 0.1% to about 0.3%, about 0.5% to about 1%, or about 0.7% to about 1%. In certain embodiments, the surfactant is added to the cell culture medium at a final concentration of about 1%. In certain embodiments, the surfactant is added to the cell culture medium at a final concentration of about 0.1%.

In certain embodiments, the final concentration of the surfactant in the culture medium is a concentration sufficient to produce a desired increased in rAAV yield. For example, in certain embodiments, the final concentration of the surfactant in the culture medium is sufficient to reduce the amount of rAAV lost during a filtration step by at least 10%, at least 20%, at least 30%, at least 40%, at least 50%, at least 60%, at least 70%, at least 80%, or at least 90%.

4. Purification of rAAV Particles

It is contemplated that the disclosed methods may, e.g., comprise any appropriate additional rAAV harvesting or purification step. In certain embodiments, rAAV particles are obtained from producer cells by lysing the cells. Lysis of producer cells can be accomplished by methods that chemically or enzymatically treat the cells in order to release infectious viral particles. These methods include the use of nucleases such as benzonase or DNAse, proteases such as trypsin, or detergents or surfactants. Physical disruption, such as homogenization or grinding, or the application of pressure via a microfluidizer pressure cell, or freeze-thaw cycles may also be used. Alternatively, supernatant may be collected from producer cells without the need for cell lysis. In certain embodiments, a disclosed method comprises a harvesting step that does not comprise a step of lysing the producer cells.

After harvesting rAAV particles, it may be necessary to purify the sample containing rAAV, to remove, for example, cellular debris. Methods of minimal purification of AAV particles are known in the art. In addition to filtration, additional exemplary purification methods are Cesium chloride (CsCl)- and iodixanol-based density gradient purification. Both methods are described in Strobel et al. (2015) Human Gene Therapy Methods 26(4):147-157 Minimal purification can also be accomplished using affinity chromatography using, for example AVB Sepharose affinity resin (GE Healthcare Bio-Sciences AB, Uppsala, Sweden). Methods of AAV purification using AVB Sepharose affinity resin are described in, for example, Wang et al. (2015) Mol. Ther. Methods Clin. Dev. 2:15040. Following purification, rAAV particles may be filtered and stored at ≤−60° C.

5. Quantification of rAAV Particles

Quantification of rAAV particles is complicated by the fact that AAV infection does not result in cytopathic effect in vitro, and therefore plaque assays cannot be used to determine infectious titers. rAAV particles can be quantified using a number of methods, however, including quantitative polymerase chain reaction (qPCR) (Clark et al. (1999) Hum. Gene Ther. 10:1031-1039) or dot-blot hybridization (Samulski et al. (1989) J. Virol. 63:3822-3828), or by optical density of highly purified vector preparations (Sommer et al. (2003) Mol. Ther. 7:122-128). DNase-resistant particles (DRP) can be quantified by real-time quantitative polymerase chain reaction (qPCR) (DRP-qPCR) in a thermocycler (for example, an iCycler iQ 96-well block format thermocycler (Bio-Rad, Hercules, Calif.)). Samples containing rAAV particles are incubated in the presence of DNase I (100 U/ml; Promega, Madison, Wis.) at 37° C. for 60 min, followed by proteinase K (Invitrogen, Carlsbad, Calif.) digestion (10 U/ml) at 50° C. for 60 min, and then denatured at 95° C. for 30 min. The primer-probe set used should be specific to a non-native portion of the rAAV vector genome, for example, the poly(A) sequence of the protein of interest. The PCR product can be amplified using any appropriate set of cycling parameters, based on the length and composition of the primers, probe, and amplified sequence. Alternative protocols are disclosed in, for example, Lock et al. (2014) Human Gene Therapy Methods 25(2): 115-125.

The infectivity of rAAV particles can be determined using a TCID50 (tissue culture infectious dose at 50%) assay, as described for example in Zhen et al. (2004) Human Gene Therapy 15:709-715. In this assay, rAAV vector particles are serially diluted and used to co-infect a Rep/Cap-expressing cell line along with AV particles in 96-well plates. 48 hours post-infection, total cellular DNA from infected and control wells is extracted. rAAV vector replication is then measured using qPCR with transgene-specific probe and primers. TCID50 infectivity per milliliter (TCID50/ml) is calculated with the Kärber equation, using the ratios of wells positive for AAV at 10-fold serial dilutions.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

Practice of the invention will be more fully understood from the foregoing examples, which are presented herein for illustrative purposes only, and should not be construed as limiting the invention in any way.

### EXAMPLES

### Example 1

This example demonstrates increased recovery of rAAV during clarification by supplementation of cell culture harvest supernatants with Triton X-100.

rAAV bulk harvest material was produced through culture of a HeLa producer cell line and infection with a helper virus in a 3 L Single-Use Bioreactor (SUB). The cells were cultured in a protein-free, chemically-defined production medium with a fraction of growth medium carried over through inoculation. The cells were cultured in 3 L bioreactors with starting volumes of 2 L and initial cell densities of 0.6×10⁶ cells/mL, and maintained at temperature and dissolved oxygen (DO) set points of 37° C. and 50%, respectively, for five days. pH was controlled using 1M sodium carbonate at set point of 7.3 for four days and elevated to 8.0 during the remainder of the experiment. The cultures were sampled daily to monitor cell growth and metabolites.

The cell culture supernatant was collected by centrifugation of 1 mL bulk harvest material at 13,000×g for 2 minutes. The cell culture supernatant was treated with or without 1% Triton X-100 prior to filtration through a 0.2 μm PES (polyethersulfone) filter.

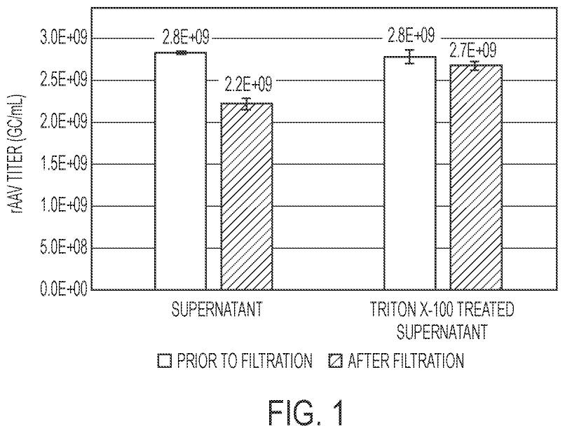

rAAV recovery was evaluated before and after the filtration step. Supernatant from the samples were digested with DNase I and then Proteinase K to liberate rAAV genomic DNA. TaqMan qPCR amplifying the BGH-PolyA coding region of the rAAV genome was then used to determine the rAAV genome copy number (GC) based on a rAAV plasmid standard curve. As depicted in FIG. 1, filtration of the supernatant through 0.2 μm PES filters resulted in a 21.4% average reduction in rAAV yield. However, this loss was completely mitigated by supplementation of the cell culture supernatant with Triton X-100.

### Example 2

This example demonstrates increased recovery of rAAV by supplementation of bulk harvest materials with Triton X-100.

rAAV bulk harvest material was produced through culture of a HeLa producer cell line and infection with a helper virus in a 50 L SUB. The rAAv bulk harvest material was treated with or without 1% Triton X-100 for 1 hour at 37° C. prior to filtration through a 0.2 μm PES filter.

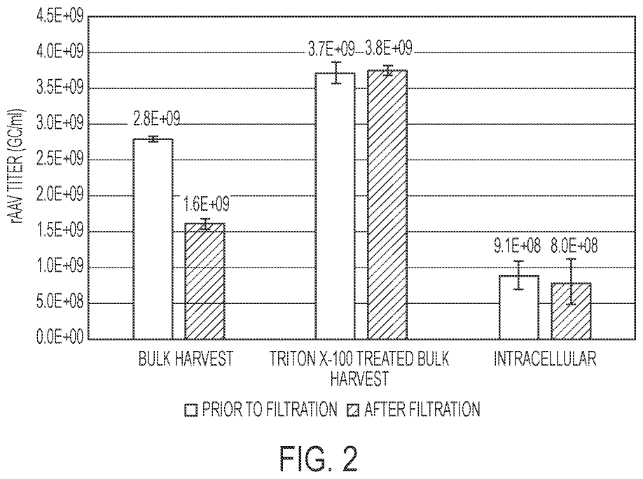

Virus recovery was evaluated by measuring viral titer by qPCR before and after the filtration step, as described in Example 1. As depicted in FIG. 2, filtration of rAAV bulk harvest material through 0.2 μm PES filters resulted in a 43% average reduction in rAAV yield. However, this loss was completely mitigated by supplementation of the cell culture supernatant with Triton X-100.

Intracellular rAAV in the bulk harvest material was determined by qPCR analysis on cells isolated by centrifugation at 13,000×g for 2 minutes. Cells were re-suspended in PBS or water, and lysed by deoxycholate. The amount of intracellular rAAV in the bulk harvest material is depicted in FIG. 2. The amount of intracellular rAAV in the bulk harvest material is similar to the amount of rAAV lost after filtration in the absence of Triton X-100.

Together, these results suggest that that rAAV recovery can be increased by supplementation of bulk harvest materials with Triton X-100, and that Triton X-100 may increase rAAV yield, in part, by contributing to increased cell lysis and intracellular rAAV release.

### Example 3

This example demonstrates increased recovery of rAAV by supplementation of bulk harvest materials with Triton X-100 at concentrations as low as 0.1%.

rAAV bulk harvest material was produced through culture of a HeLa producer cell line and infection with a helper virus in a 250 ml Erlenmeyer flask. The rAAV bulk harvest material was treated with 0%, 0.01%, 0.1% or 1% Triton X-100 for 1 hour at 37° C. prior to filtration through a 0.2 μm PES filter.

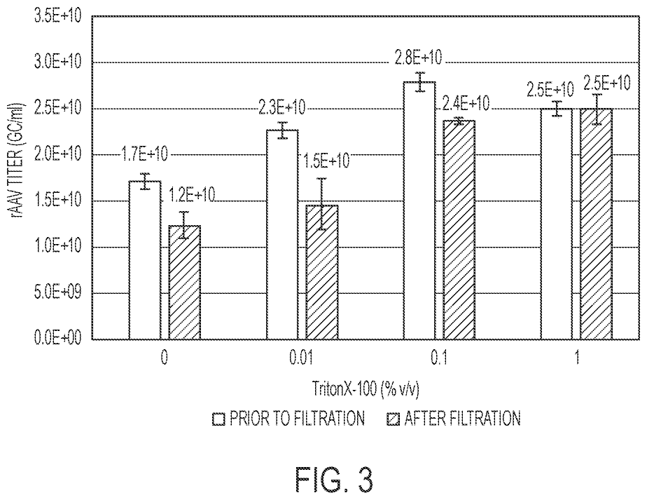

Virus recovery after filtration was evaluated by measuring viral titer by qPCR before and after the filtration step. As depicted in FIG. 3, filtration of rAAV bulk harvest material through 0.2 μm PES filters resulted in a significant reduction of rAAV yield from bulk harvest material treated with 0 or 0.01% Triton X-100. However, this loss was completely mitigated by supplementation of the bulk harvest material with 0.1% or 1% Triton X-100.

### Example 4

This example demonstrates increased recovery of rAAV during clarification by supplementation of cell culture harvest supernatants with Triton X-100.

rAAV bulk harvest material was produced through culture of a HeLa producer cell line and infection with a helper virus in a 50 L SUB.

The cell culture supernatant was collected by centrifugation of 1 mL bulk harvest material at 13,000×g for 2 minutes. The cell culture supernatant was treated with or without 1% Triton X-100 prior to a first and second filtration through 0.2 μm PES filters.

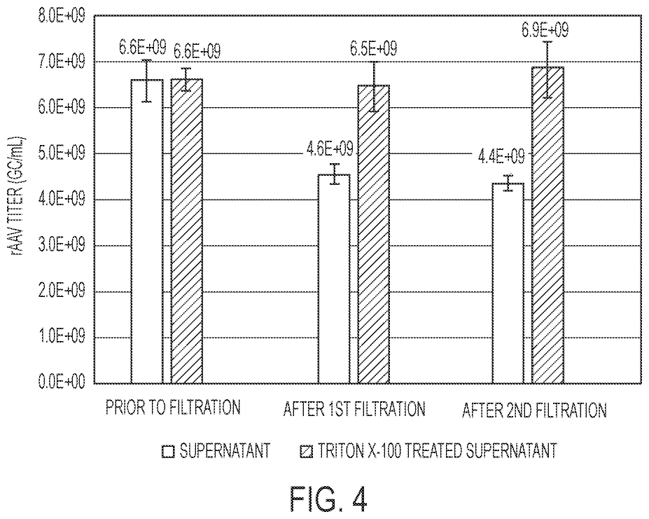

rAAV recovery after the first filtration was evaluated by measuring viral titer by qPCR before and after the filtration step. As shown in FIG. 4, filtration of rAAV cell culture supernatant through 0.2 μm PES filters resulted in a 30% average reduction in yield. However, this loss was completely mitigated by supplementation of the cell culture supernatant with Triton X-100.

Virus recovery after the second filtration was evaluated by measuring viral titer by qPCR before and after the second filtration step. As depicted in FIG. 4, subsequent filtration through a second 0.2 μm PES filter resulted in no additional losses in viral titer, with or without Triton X-100 supplementation. These results suggest that rAAV does not bind PES filters alone, reductions in rAAV yield may be due to adherence of rAAV to light weight cell debris or viral aggregates, and Triton X-100 may detach rAAV from cell debris or dissociate aggregated virus, resulting in complete recovery following filtration.

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. A method of producing a clarified recombinant adeno-associated virus vector (rAAV) product comprising the steps of:
(a) incubating a plurality of producer cells in a cell culture medium under conditions that promote release of rAAV particles, whereby rAAV particles are released from the producer cells into the culture medium, and the producer cells are not substantially lysed;
(b) recovering the cell culture medium from the producer cells, so that the recovered cell culture medium is substantially free of intact producer cells and contains rAAV particles;
(c) adding a surfactant to the recovered cell culture medium, wherein the surfactant is a non-ionic surfactant with a hydrophilic-lipophilic balance (HLB) between 12 and 15; and
(d) passing the recovered cell culture medium containing the surfactant through a first filter;
wherein the method does not comprise a step of lysing the producer cells.

2. The method of claim 1, wherein the step of recovering the cell culture medium from the producer cells, so that the recovered cell culture medium is substantially free of intact producer cells and contains rAAV particles, comprises the sub-steps of:
(i.) centrifuging the producer cells in the cell culture medium to create a cell pellet; and
(ii.) separating the cell culture medium from the cell pellet, and retaining the cell culture medium.

3. The method of claim 2, wherein the producer cells in the cell culture medium are centrifuged at a speed of greater than 150×g or 300×g.

4. The method of claim 2 or 3, wherein the producer cells in the cell culture medium are centrifuged at a speed of less than 500×g or 13,000×g.

5. The method of any preceding claim, wherein the step of recovering the cell culture medium from the producer cells, so that the recovered cell culture medium is substantially free of intact producer cells and contains rAAV particles, comprises the sub-step of aspirating the cell culture medium into a vessel.

6. The method of any preceding claim, wherein the step of recovering the cell culture medium from the producer cells, so that the recovered cell culture medium is substantially free of intact producer cells and contains rAAV particles, comprises the sub-step of changing the pH of the cell culture medium.

7. A method of producing a clarified recombinant adeno-associated virus vector (rAAV) product comprising the steps of:
(a) incubating a plurality of producer cells in a cell culture medium under conditions that promote release of rAAV particles, whereby rAAV particles are released from the producer cells into the culture medium, and the producer cells are not substantially lysed;
(b) adding a surfactant to the cell culture medium, wherein the surfactant is a non-ionic surfactant with a hydrophilic-lipophilic balance (HLB) between 12 and 15;
(c) incubating the producer cells in the surfactant-containing cell culture medium for a time, the time being insufficient to cause the lysis of 50% of the producer cells; and
(d) passing the cell culture medium containing the surfactant through a first filter.

8. The method of claim 7, wherein incubation in the surfactant-containing cell culture medium results in lysis of fewer than 40% of the producer cells.

9. The method of claim 8, wherein incubation in the surfactant-containing cell culture medium results in lysis of fewer than 30% of the producer cells.

10. The method of claim 9, wherein incubation in the surfactant-containing cell culture medium results in lysis of fewer than 10% of the producer cells.

11. The method of claim 10, wherein incubation in the surfactant-containing cell culture medium results in lysis of fewer than 1% of the producer cells.

12. The method of any preceding claim, wherein release of the rAAV particles from the producer cells into the culture medium results in lysis of fewer than 50% of the producer cells.

13. The method of any preceding claim, wherein release of the rAAV particles from the producer cells into the culture medium results in lysis of fewer than 30% of the producer cells.

14. The method of any preceding claim, wherein release of the rAAV particles from the producer cells into the culture medium results in lysis of fewer than 10% of the producer cells.

15. The method of any preceding claim, wherein release of the rAAV particles from the producer cells into the culture medium results in lysis of fewer than 1% of the producer cells.

16. The method of any preceding claim, wherein the surfactant is added to the cell culture medium at a final concentration of about 1% to about 0.01%.

17. The method of claim 16, wherein the surfactant is added to the cell culture medium at a final concentration of about 0.1% to about 0.5%.

18. The method of claim 16, wherein the surfactant is added to the cell culture medium at a final concentration of about 0.1% to about 0.3%.

19. The method of claim 16, wherein the surfactant is added to the cell culture medium at a final concentration of about 0.5% to about 1%.

20. The method of claim 16, wherein the surfactant is added to the cell culture medium at a final concentration of about 0.7% to about 1%.

21. The method of claim 16, wherein the surfactant is added to the cell culture medium at a final concentration of about 1%.

22. The method of claim 16, wherein the surfactant is added to the cell culture medium at a final concentration of about 0.1%.

23. The method of any preceding claim, further comprising step (e), passing the cell culture medium containing the surfactant through a second filter.

24. The method of any preceding claim, wherein one or more of the filters are selected from the group consisting of a PES membrane filter, a PVDF membrane filter, a nylon membrane filter, and a MCE membrane filter.

25. The method of claim 24, wherein one or more of the filters is a PES membrane filter.

26. The method of any preceding claim, wherein the filter has a pore size of less than 1 μm.

27. The method of claim 26, wherein the filter has a pore size of less than 0.5 μm.

28. The method of claim 27, wherein the filter has a pore size of 0.22 μm.

29. The method of any preceding claim, wherein the concentration of rAAV particles in the rAAV-containing cell culture medium after the first filtration step is greater than 80% of the concentration of rAAV particles in the rAAV-containing cell culture medium immediately prior to the first filtration step.

30. The method of claim 29, wherein the concentration of rAAV particles in the rAAV-containing cell culture medium after the first filtration step is greater than 90% of the concentration of rAAV particles in the rAAV-containing cell culture medium immediately prior to the first filtration step.

31. The method of claim 30, wherein the concentration of rAAV particles in the rAAV-containing cell culture medium after the first filtration step is greater than 95% of the concentration of rAAV particles in the rAAV-containing cell culture medium immediately prior to the first filtration step.

32. The method of claim 31, wherein the concentration of rAAV particles in the rAAV-containing cell culture medium after the first filtration step is greater than 99% of the concentration of rAAV particles in the rAAV-containing cell culture medium immediately prior to the first filtration step.

33. The method of any preceding claim, wherein the surfactant is an octylphenol ethoxylate.

34. The method of claim 33, wherein the surfactant is an octylphenol ethoxylate with an HLB between 13 and 14.

35. The method of claim 33, wherein the surfactant is an octylphenol ethoxylate with an average polyethylene oxide chain length between 8 and 12.

36. The method of claim 33, wherein the surfactant is an octylphenol ethoxylate with an average polyethylene oxide chain length between 9 and 10.

37. The method of any preceding claim, wherein the producer cells are mammalian cells.

38. The method of claim 37, wherein the producer cells are selected from the group consisting of HeLa, HEK293, COS, A549, and Vero cells.

39. The method of claim 38, wherein the producer cells are HeLa cells.

40. The method of any one of claims 1-36, wherein the producer cells are insect cells.

41. The method of claim 40, wherein the insect cells are selected from the group consisting of Sf9, Sf-21, Tn-368, and BTI-Tn-5B1-4 (High-Five) cells.

42. A rAAV produced by the method of any preceding claim.
